(19) United States
(12) Patent Application Publication
(10) Pub. No.: US 2026/0090724 A1
(43) Pub. Date: Apr. 2, 2026

(54) METHOD AND TERMINAL DEVICE FOR PROVIDING TOOTH DETERIORATION INFORMATION TO USER

(71) Applicant: SMARTOOTH CO., LTD., Seoul (KR)

(72) Inventors: Ho Jung SON, Seoul (KR); Myung Seon RYOU, Seoul (KR); Dong-Woo LEE, Gimpo-si (KR)

(21) Appl. No.: 19/345,058

(22) Filed: Sep. 30, 2025

Related U.S. Application Data
(63) Continuation-in-part of application No. 17/991,265, filed on Nov. 21, 2022.
(63) Continuation-in-part of application No. 18/789,117, filed on Jul. 30, 2024.

(30) Foreign Application Priority Data
May 11, 2022 (KR) 10-2022-0057897
Oct. 17, 2023 (KR) 10-2023-0138287

(51) Int. Cl. A61B 5/00

(52) U.S. Cl. CPC A61B 5/0088; A61B 5/742

(57) ABSTRACT

A method for diagnosing conditions of teeth includes irradiating light to each tooth of a user; receiving the light reflected from the each tooth; determining a first tooth state numerical value for the each tooth of the user; determining a second tooth state numerical value for the each tooth of the user; selecting at least one target tooth which was not treated in the non-measurement period; calculating a difference between the second tooth state numerical value and the first tooth state numerical value for each of the at least one target tooth; calculating a weighted difference for each of the at least one target tooth based on the difference and a weight assigned for each of the at least one target tooth; and obtaining tooth deterioration information of the user at the second measurement period based on the weighted difference for each of the at least one target tooth.

### CROSS-REFERENCE TO RELATED APPLICATIONS

This application is a Continuation in-part of application Ser. No. 17/991,265 filed on Nov. 21, 2022, which claims priority to Korean Patent Application No. 10-2022-0057897 filed on May 11, 2022. This application is also a Continuation in-part of application Ser. No. 18/789,117 filed on Jul. 30, 2024, which claims priority to Korean Patent Application No. 10-2023-0138287 filed on Oct. 17, 2023. The aforementioned applications are incorporated herein by reference in their entireties.

### BACKGROUND

### 1. Technical Field

Embodiments of the present invention relate to a method of managing teeth capable of providing a user with state deterioration numerical information on his or her teeth, and a terminal device for performing the same.

### 2. Related Art

When a user does not properly brush or does not use floss after eating food, food residues remain on teeth, which may lead to the proliferation of bacteria, and thus, infectious diseases such as tooth decay.

In order to prevent the above-described oral diseases and keep teeth healthy, it is important to periodically measure a user's tooth state to observe the tooth state. Accordingly, there is a need for a technology that can simply and easily check the tooth state, and provide users with meaningful notification information related to teeth based on the tooth state.

### SUMMARY

The object of the present invention is to provide a method of managing teeth capable of periodically collecting user's teeth-related state information measured by a tooth diagnostic device, and providing simply and easily a tooth state to a user based on the periodically collected tooth information, and a terminal device for performing the same.

In addition, the object of the present invention is to provide a method of managing teeth for providing tooth-related meaningful information, in particular, deterioration numerical information on a tooth to a user based on a measured tooth state, and a terminal device for performing the same.

In addition, the object of the present invention is to provide a method of managing teeth capable of providing genetic predisposition information on the occurrence of caries to a user, and a terminal device for performing the same.

In addition, the object of the present invention is to provide a method of managing teeth capable of performing tooth management for each management group (e.g., daycare center, kindergarten, nursing hospital, etc.) including a plurality of users, and providing comparison information related to the management group to the user, and a terminal device for performing the same.

The objects of the present invention are not limited to the above-described objects, and other objects and advantages of the present invention that are not mentioned may be understood by the following description and will be more clearly understood by embodiments of the present invention. In addition, it may be easily appreciated that the objects and advantages of the present invention may be realized by means mentioned in the claims and a combination thereof.

According to an aspect of the present invention, a method of managing teeth performed in a processor-based device includes: calculating tooth deterioration information of the user in a second measurement period based on a first tooth state numerical value of each of user's teeth in a first measurement period and a second tooth state numerical value of each of the user's teeth in the second measurement period after the first measurement period; and providing the tooth deterioration information to the user.

According to an embodiment of the present invention, an oral diagnostic device includes a device main body that includes an optical transmitting and receiving module, and a probe that is detachably coupled to the device main body, irradiates irradiated light received from the optical transmitting and receiving module into an oral cavity, receives reflected light from the irradiated light reflected from the oral cavity, and transmits the received reflected light to the optical transmitting and receiving device. In this case, the optical transmitting and receiving module includes an optical element module including a light-emitting element and a light-receiving element, a single first optical fiber that transmits the irradiated light generated by the light-emitting element to the probe, and a plurality of second optical fibers that transmit the reflected light received from the probe to the light-receiving element, and the other side of the single first optical fiber and the other side of the plurality of second optical fibers are coupled to each other to form an optical fiber coupling part.

According to another aspect of the present invention, a terminal device includes: a memory configured to store a computer-readable instruction; a processor implemented to execute the instruction; and a display unit configured to display an execution result of the instruction, in which the processor calculates tooth deterioration information of the user in a second measurement period based on a first tooth state numerical value of each of user's teeth in a first measurement period and a second tooth state numerical value of each of the user's teeth in the second measurement period after the first measurement period, and the display unit displays the tooth deterioration information.

### BRIEF DESCRIPTION OF THE DRAWINGS

FIG. 1 is a diagram illustrating a schematic configuration of a system for managing teeth according to an embodiment of the present invention.

FIG. 2 is a block diagram illustrating a schematic configuration of a tooth diagnostic device according to an embodiment of the present invention.

FIG. 3 is a block diagram illustrating a schematic configuration of a user terminal according to an embodiment of the present invention.

FIGS. 4-6 are diagrams illustrating a flowchart of a method of managing teeth according to an embodiment of the present invention.

FIG. 7 is a diagram illustrating a tooth state numerical value measured in first and second measurement periods for explaining the embodiments of the present invention.

FIG. 8 is a perspective view of an oral diagnostic device according to an embodiment of the present invention.

FIG. 9 is a front view of the oral diagnosis device according to the embodiment of the present invention.

FIG. 10 is an exploded perspective view of the oral diagnosis device according to the embodiment of the present invention.

FIGS. 11A and 11B are diagrams illustrating a state in which the oral diagnosis device according to the embodiment of the present invention is used.

FIG. 12 is a diagram illustrating a detailed configuration of a device main body according to the embodiment of the present invention.

FIG. 13 is a perspective view of an optical transmitting and receiving module according to an embodiment of the present invention.

FIG. 14 is a cross-sectional view of the optical transmission/reception module according to the embodiment of the present invention.

FIG. 15 is a diagram illustrating a plan shape of the optical transmitting and receiving module installed in a main body case according to an embodiment of the present invention.

FIG. 16 is a perspective view of a probe according to an embodiment of the present invention.

FIG. 17 is a diagram illustrating a combined shape of the optical transmitting and receiving module and the probe according to the embodiment of the present invention.

### DETAILED DESCRIPTION

The present invention may be variously modified and have several embodiments, and thus, specific embodiments will be illustrated in the accompanying drawings and be described in detail. However, it is to be understood that the present invention is not limited to a specific exemplary embodiment, but includes all modifications, equivalents, and substitutions included in the scope and spirit of the present invention. In describing each drawing, similar reference numerals are used for similar components.

The terms such as “first,” “second,” or the like, may be used to describe various components, but these components are not to be construed as being limited to these terms. The terms are used only to distinguish one component from another component. The term “and/or” includes a combination of a plurality of related described items or any one of the plurality of related described items.

The terms used in the present specification are used only to describe specific embodiments rather than limiting the present invention. Singular forms are intended to include plural forms unless the context clearly indicates otherwise. It is to be understood that the term “include” or “have” used herein specifies the presence of features, numbers, steps, operations, components, parts, or combinations thereof mentioned in the present specification, or combinations thereof, but does not preclude the presence or addition of one or more other features, numbers, steps, operations, components, parts, or combinations thereof.

Hereinafter, embodiments of the present invention will be described in detail with reference to the accompanying drawings.

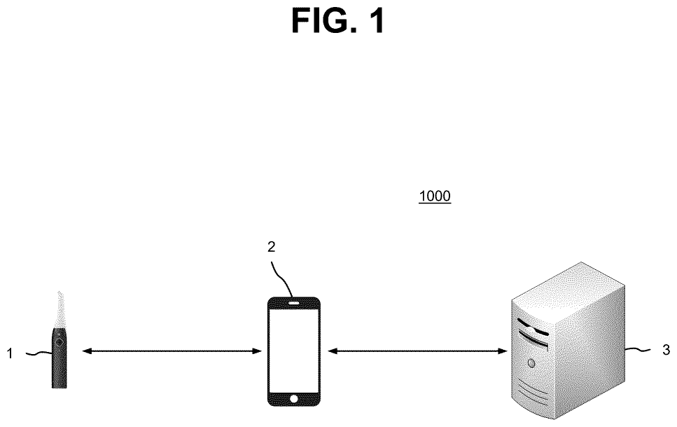

FIG. 1 is a diagram illustrating a schematic configuration of a system 1000 for managing teeth according to an embodiment of the present invention.

Referring to FIG. 1, the system 1000 for managing teeth according to an embodiment of the present invention may include a tooth diagnostic device (herein interchangeably referred to as an oral diagnostic device) 1, a user terminal 20, and a tooth management server 30.

The tooth diagnostic device 1 may be a device for measuring a state of a user's teeth and/or an interdental region. For example, the tooth diagnostic device 1 may be a portable device that measures a state of user's teeth and/or interdental region using an optical sensor. However, the present invention is not limited thereto, and the present invention may be applied to any type of tooth diagnostic device 1. For example, the tooth diagnostic device 1 includes a tooth diagnostic device of an acidity (pH) measurement method, a camera-based tooth diagnostic device, and the tooth diagnostic device 1 interlocked with X-rays.

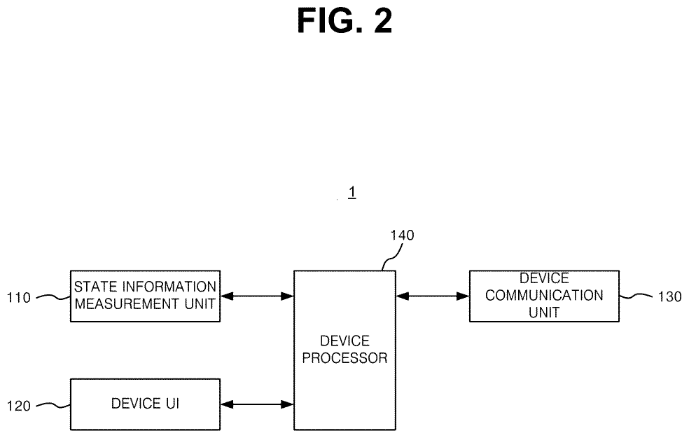

FIG. 2 is a block diagram illustrating a schematic configuration of the tooth diagnostic device 1 according to an embodiment of the present invention.

Referring to FIG. 2, the tooth diagnostic device 1 may include a state information measurement unit 110, a user interface unit 120, a device communication unit 130, and a device processor 140.

The state information measurement unit 110 may include an optical sensor that measures the state of the user's teeth and/or the interdental region based on transmission/reception of light (e.g., laser). Alternatively, the state information measurement unit 110 may correspond to a probe including a pH sensor for measuring the acidity (pH) of the user's teeth and/or the interdental region.

Hereinafter, for convenience of description, the “state information of the tooth and/or the interdental region” will be referred to as “teeth-related state information.”

The device user interface (UI) 120 may receive an input signal from a user, and may display various types of information related to the state information measurement unit 110.

The device communication unit 130 may be a communication module that communicates with an external electronic device. In particular, the device communication unit 130 may transmit the teeth-related state information to the user terminal 20. To this end, the device communication unit 130 may include a short-range communication module. In particular, the device communication unit 130 may include a Bluetooth module.

The device processor 140 may include one or more of a central processing unit including an internal memory, an application processor, or a communication processor. The device processor 140 may execute an operation or data processing related to control and/or communication of at least one other component of the tooth diagnostic device 1. In particular, the device processor 140 may generate the state information of the tooth and/or the interdental region measured by the state information measurement unit 110.

The teeth-related state information may correspond to a state numerical value (or a decay value). For example, the state numerical value may have any one numerical value of 0 to 99 points. The higher the state numerical value, the more cavities of the teeth and/or the interdental region may progress, and the lower the state numerical value, the healthier the teeth and/or the interdental region.

According to an embodiment, the teeth-related state information may be represented in a plurality of stages. The plurality of stages may include a first stage, a second stage, and a third stage.

The first stage may be a caries good stage. The caries good stage may be a stage in which teeth are hardly damaged. For example, a section of the state numerical value corresponding to the first stage may be 0 to 19 points.

The second stage may be a caries attention stage. The caries attention stage may be a stage in which enamel of a tooth is damaged. For example, a section of the state numerical value corresponding to the second stage may be 20 to 39 points.

The third stage may be a caries risk stage. The caries risk stage may be a stage in which enamel and dentin of a tooth are damaged. For example, a section of the state numerical value corresponding to the third stage may be 40 to 99 points.

Referring to the above, the tooth diagnostic device 1 may calculate teeth-related state information (state numerical value) based on information measured by an optical sensor or a pH sensor, and transmit the calculated teeth-related state information of the user to the terminal 20. However, the present invention is not limited thereto. According to another embodiment, the tooth diagnostic device 1 may transmit the information measured by the optical sensor or the pH sensor to the user terminal 20, and the user terminal 20 may calculate the teeth-related state information (state numerical value) based on the information measured by the optical sensor or the pH sensor.

Referring back to FIG. 1, the user terminal 20 may be a terminal owned by a user. For example, the user terminal 20 may be a smart device such as a smart phone, a smart pad, a smart location, or a smart TV.

If the user is an adult, the user terminal 20 may be a terminal owned by the user. If the user is a child, the user terminal 20 may be a terminal possessed by the user's parents.

The user terminal 20 may receive the user's teeth-related state information measured by the tooth diagnostic device 1, and may generate user's oral result state information based on the received teeth-related state information. The oral result state information may be visually output to a user.

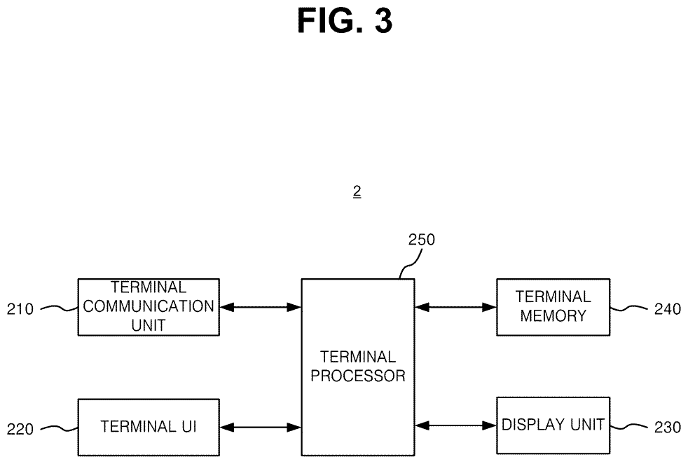

FIG. 3 is a block diagram illustrating a schematic configuration of the user terminal 20 according to the embodiment of the present invention.

Referring to FIG. 3, the user terminal 20 may include a terminal communication unit 210, a terminal UI 220, a display unit 230, a terminal memory 240, and a terminal processor 250.

The terminal communication unit 210 may be a communication module for performing communication with the tooth diagnostic device 1 and the tooth management server 30. To this end, the terminal communication unit 210 may include a first terminal communication module and a second terminal communication module.

The first terminal communication module may communicate with the tooth diagnostic device 1. As an example, the first terminal communication module may be a short-range communication module, in particular, a Bluetooth module.

The second terminal communication module may communicate with the tooth management server 30. For example, the second terminal communication module may be a short-range communication module (e.g., a WiFi module) and/or a long-distance communication module.

The terminal UI 220 may receive an input signal from a user. The input signal may include a text signal, a voice signal, and a touch event signal.

The display unit 230 may be a device capable of displaying an image or an image frame to a user. The display unit 230 may output an execution screen of an application executed by the terminal processor 250 to be described later, and in particular, may visually display various tooth images.

Meanwhile, the terminal UI 220 and the display unit 230 may be implemented as one device. That is, the display unit 230 may be a touch display that receives a user's touch signal, and in this case, the terminal UI 220 may be omitted from the user terminal 20.

The terminal memory 240 may be a volatile and/or non-volatile memory, and may store instructions or data related to at least one other component of the user terminal 20. In particular, the terminal memory 240 may store instructions or data related to an application (a computer program or a recording medium) executed in the user terminal 20.

In addition, the terminal memory 240 may store information related to a user (i.e., user information) and information related to management of a tooth. Here, the user information may be inputted from the user and collected, and the information related to the management of the tooth may be collected from the outside through the terminal communication unit 210. The user information may include information on the number of teeth of a user. The information on the number of teeth of the user may include information on the number of teeth not existing in a user's oral cavity.

The terminal processor 250 may include one or more of a central processing unit, an application processor, and a communication processor. The terminal processor 250 may execute an operation or data processing related to control and/or communication of at least one other component of the user terminal 20. In particular, the terminal processor 250 may execute instructions of an application.

Referring back to FIG. 1, the tooth management server 30 may receive and store various types of information stored in the user terminal 20. To this end, the tooth management server 30 may include a communication unit, a memory, and a processor.

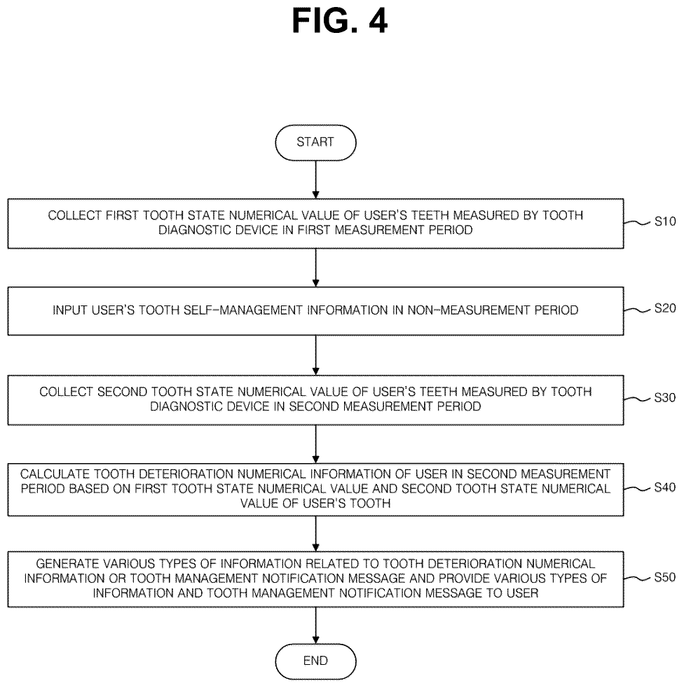

Hereinafter, a method of managing teeth will be described in detail with reference to FIGS. 4 to 7.

FIG. 4 is a diagram illustrating an overall flowchart of a method of managing teeth according to an embodiment of the present invention.

In this case, all steps of the method of managing teeth illustrated in FIG. 4 may be performed by the user terminal 20. Meanwhile, some steps of the method of managing teeth illustrated in FIG. 4 may be performed by a tooth management server 30. In this case, some steps may be steps of generating information or a notification message to a user in steps S10 to S40 and S50, which will be described later.

Hereinafter, for convenience of description, it is assumed that the method of managing teeth is performed in the user terminal 20.

In addition, it is assumed that the user terminal 20 is a smart phone having the touch display unit 230. In addition, it is assumed that the method of managing teeth is performed in a tooth management application executed in the user terminal 20, and the user terminal 20 has completed a communication connection (e.g., Bluetooth pairing) with the tooth diagnostic device 1. In addition, it is assumed that the tooth state information is a tooth state numerical value.

Hereinafter, the process performed for each step will be described in detail.

In step S10, the user terminal 20 may collect a first tooth state numerical value of user's teeth measured by the tooth diagnostic device 1 in the first measurement period.

The first measurement period may be defined as a measurement period in which the state numerical values of the user's teeth are first measured. The first measurement period may be a period corresponding to a specific day.

In step S20, the user terminal 20 may receive user's tooth self-management information from a user in the non-measurement period.

Here, the non-measurement period is a period that arrives after the first measurement period, and may be a period composed of a plurality of days. For example, the non-measurement period may be “15 days”, but the present invention is not limited thereto.

During the non-measurement period, the tooth state numerical value may not be received by the tooth diagnostic device 1. That is, the non-measurement period may be a time interval between the first measurement period and a second measurement time to be described later.

The tooth self-management information may be input through the touch display unit 230 of the user terminal 20. That is, in the non-measurement period, a user may input management information of teeth performed by himself or herself to the user terminal 20.

According to the embodiment, the tooth self-management information may include at least one of brushing information, toothbrush type information during brushing, and floss use information that are performed by the user in the non-measurement period.

Meanwhile, the tooth self-management information may be input to the user terminal 20 on a daily basis, for example. In addition, when the user does not self-manage teeth on a specific day, the user may not input the tooth self-management information to the user terminal 20. That is, the user terminal 20 may receive the tooth self-management information only on a specific day when a user self-manages teeth.

In order to receive the tooth self-management information from the user, the user terminal 20 may output (i.e., display) a guidance message for inducing the user's tooth self-management in the non-measurement period to the user. The guidance messages may also be output to the user terminal 20 on a daily basis. The user may perform self-management of teeth after checking the guidance message. The tooth self-management information may correspond to a response of the guidance message. As an example, the guidance messages may be “Did you brush your teeth today?”, “Did you use dental floss between molars which are a caries attention stage?” etc.

In step S30, the user terminal 20 may collect a second tooth state numerical value of user's teeth measured by the tooth diagnostic device 1 in the second measurement period.

The second measurement period may be a period that arrives after the non-measurement period. That is, the second measurement period may be a measurement period in which a user's tooth state numerical value is collected in the tooth diagnostic device 1 after the first measurement period. The second measurement period may be a period corresponding to a specific day.

Meanwhile, the number of teeth for which the second tooth state numerical value is measured in the second measurement period and the number of teeth for which the first tooth state numerical value is measured in the second measurement period may be the same or different.

In step S40, the user terminal 20 may calculate the user's deterioration numerical information in the second measurement period based on the first tooth state numerical value and the second tooth state numerical value of the user's teeth. This may be performed in the terminal processor 250.

Here, the tooth deterioration information is defined as information indicating how much worse the user's tooth state is than the user's tooth state in the first measurement period. The tooth deterioration information may correspond to the tooth deterioration numerical information or tooth deterioration numerical value. Hereinafter, for convenience of description, the present embodiments will be described assuming that the “tooth deterioration information” is the “tooth deterioration numerical information” or “tooth deterioration numerical value”.

Meanwhile, in general, the tooth state may not be improved naturally without treatment in a hospital. Accordingly, the tooth deterioration numerical value may not have a negative value and may have a value greater than or equal to zero. In addition, as the tooth state worsens over time, the tooth deterioration numerical value may increase.

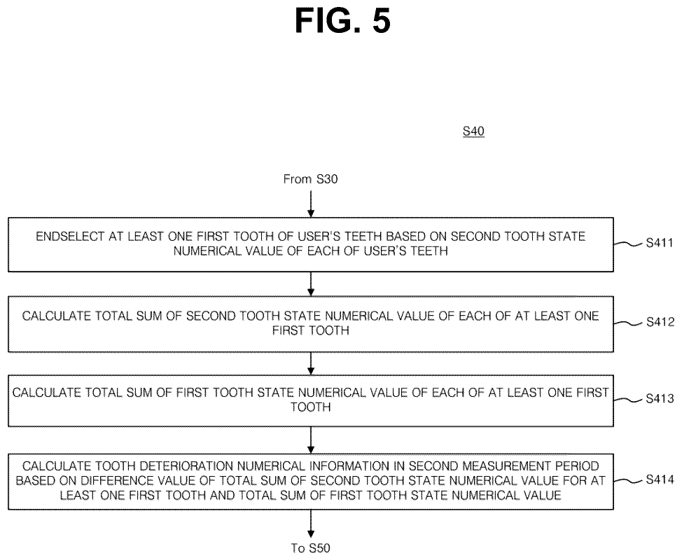

Hereinafter, embodiments of step S40 will be described in more detail with reference to FIGS. 5 to 7.

FIG. 5 is a diagram illustrating a flowchart of an embodiment of the above-described step S40.

Referring to FIG. 5, in step S40, the user terminal 20 may select at least one first tooth among the user's teeth based on the second tooth state numerical value of each of the user's teeth, and calculate the tooth deterioration information by comparing the second tooth state numerical value of each of the at least one first tooth with the first tooth state numerical value of each of the at least one first tooth. A detailed description thereof will be provided below.

In step S411, the user terminal 20 may select at least one first tooth among the user's teeth based on the second tooth state numerical value of each of the user's teeth.

According to the embodiment, the user terminal 20 may select at least one first tooth having the second tooth state numerical value greater than or equal to a preset tooth state threshold numerical value among the user's teeth.

In this case, the tooth state threshold numerical value may correspond to a minimum tooth state numerical value that damages the enamel of teeth.

As an example, as described above, the tooth state numerical value may be included in any one of a caries good stage (first stage, 0 to 24 points), a caries attention stage (second stage, 25 to 35 points), and a caries risk stage (third stage, 36 to 99 points), and the tooth state threshold numerical value may be 25 points, which is the minimum numerical value (i.e., the minimum tooth state numerical value that damages the enamel of teeth) of the caries attention stage. As another example, the tooth state threshold numerical value may be 36 points, which is the minimum numerical value (i.e., the minimum tooth state numerical value that damages the dentin of teeth) of the caries risk stage.

In step S412, the user terminal 20 may calculate the total sum of the second tooth state numerical values of each of the at least one first tooth.

In step S413, the user terminal 20 may calculate the total sum of the first tooth state numerical values of each of the at least one first tooth.

In step S414, the user terminal 20 may calculate the tooth deterioration information in the second measurement period based on the difference value of the total sum of the first tooth state numerical values and the total sum of the second tooth state numerical values for at least one first tooth.

As an example, the tooth deterioration information may be a simple difference value (i.e., an unweighted difference value) of the total sum of second tooth state numerical values and the total sum of first tooth state numerical values, and may be the weight-reflected difference value of the total sum of second tooth state numerical values and the total sum of first tooth state numerical values.

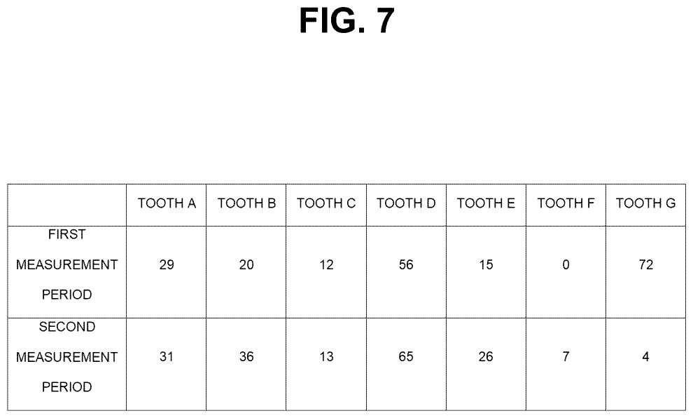

Hereinafter, an embodiment of step S40 described in FIG. 5 will be described in detail with reference to FIG. 7.

Referring to FIG. 7, the first tooth state numerical values of teeth A, B, C, D, E, and G, which are six teeth, are measured in the first measurement period. Meanwhile, in the first measurement period, the tooth state of the tooth F was not measured, and accordingly, the tooth F may have the first tooth state numerical value of the default value (=0).

The second tooth state numerical values of teeth A, B, C, D, E, F, and G, which are seven teeth, are measured in the second measurement period. In the case of the tooth G, the second tooth state numerical value is less than the first tooth state numerical value. Therefore, it may be inferred that the tooth G was treated in a hospital.

A description of how the user terminal 20 calculates the tooth deterioration information will be given based on the above description. In this case, it is assumed that the tooth state threshold numerical value is 25 points, and the tooth deterioration information is a simple difference value of the total sum of the second tooth state numerical values and the total sum of the first tooth state numerical values.

First, the user terminal 20 may select four teeth (teeth A, B, D, and E) having a second tooth state numerical value of seven teeth greater than or equal to the tooth state threshold numerical value (=25) as a first tooth.

Next, the user terminal 20 may calculate the total sum (=158) of the second tooth state numerical value of each of the four first teeth (teeth A, B, D, and E).

To be continued, the user terminal 20 may calculate the total sum (=120) of the first tooth state numerical value of each of four first teeth (teeth A, B, D, and E).

Finally, the user terminal 20 may calculate the difference value (=38) of the total sum (=158) of second tooth state numerical values and the total sum (=120) of first tooth state numerical values as the tooth deterioration numerical value.

In other words, according to an embodiment of step S40, when teeth in a caries normal stage are used for calculating the tooth deterioration numerical value, the tooth deterioration numerical value may not accurately reflect the actual deterioration degree of user's teeth. Accordingly, the user terminal 20 may use only the first tooth, which is a tooth belonging to the caries attention stage and the caries risk stage, for calculating the tooth deterioration numerical value, and may not use a tooth, which belongs to the caries normal stage, for calculating the tooth deterioration numerical value.

In addition, as described above, the tooth deterioration numerical value is defined so that it may not have a negative value. On the other hand, when the tooth F treated at the hospital is used for calculating the tooth deterioration numerical value, the tooth deterioration numerical value has a negative value. Therefore, in order to prevent an error in the calculation of the tooth deterioration numerical value and accurately calculate the tooth deterioration numerical value, teeth treated at a hospital in a non-measurement period may not be used in the calculation of the tooth deterioration numerical value.

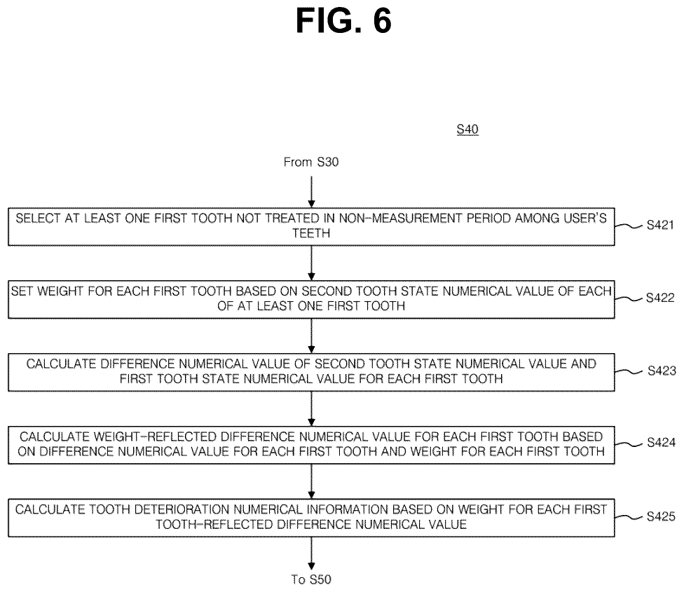

FIG. 6 is a diagram illustrating a flowchart of another embodiment of the above-described step S40.

Hereinafter, each step of FIG. 6 will be described with reference to FIG. 7 together.

In step S421, the user terminal 20 may select at least one first tooth that is not treated in the non-measurement period among the user's teeth.

As described above, teeth treated at a hospital in the non-measurement period may not be used for the calculation of the tooth deterioration numerical values. In this case, the information of the treated tooth may be directly input to the touch display 230 by the user, and may also be checked by comparing the first tooth state numerical value with the second tooth state numerical value of each of the teeth.

Referring to FIG. 7, the user terminal 20 may select, as the first teeth, six teeth (teeth A, B, C, D, E, and F) that have not been treated at a hospital among seven teeth.

In step S422, the user terminal 20 may set weights for each first tooth based on the second tooth state numerical values of each of the at least one first tooth. In this case, the weights may be the same or different for each first tooth.

According to an embodiment, when the second tooth state numerical value of the first tooth is below a preset first tooth state threshold numerical value, the weight of the first tooth may be the first weight. In addition, when the second tooth state numerical value of the first tooth is greater than or equal to the first tooth state threshold numerical value, the weight of the first tooth may be the second weight.

In this case, the second weight may be set to be greater than the first weight. In particular, the first weight may have a very small value, for example, a value of zero. Also, the first tooth state threshold numerical value may be the minimum numerical value (i.e., a minimum tooth state numerical value that damages enamel of a tooth) of the caries attention stage.

As an example, referring to FIG. 7, when the first tooth state threshold numerical value is set to 25 points, the weights of the teeth C and F may have relatively small first weights, and the weights of the teeth A, B, D, and E may have a relatively large second weight.

According to another embodiment, when the second tooth state numerical value of the first tooth is below the first tooth state threshold numerical value, the weight of the first tooth may be the first weight. In addition, when the second tooth state numerical value of the first tooth is greater than or equal to the first tooth state threshold numerical value and below the preset second tooth state threshold numerical value, the weight of the first tooth may be the second weight. In addition, when the second tooth state numerical value of the first tooth is greater than or equal to the second tooth state threshold numerical value, the weight of the first tooth may be a third weight.

In this case, the second weight may be set to be greater than the first weight, and the third weight may be set to be greater than the second weight. In particular, the first weight may have a very small value, for example, a value of zero. Also, the second tooth state threshold numerical value may be the minimum numerical value (i.e., a minimum tooth state numerical value that damages enamel of a tooth) of the caries risk stage.

As an example, referring to FIG. 7, when the first tooth state threshold numerical value is set to 25 points and the second tooth state threshold numerical value is set to 36 points, weights w_{C} and w_{F} of the teeth C and F may have the first weight, weights w_{A} and w_{E} of the teeth A and E may have the second weight, and weights w_{B} and w_{D} of the teeth B and D may have the third weight.

In step S423, the user terminal 20 may calculate a difference numerical value between the second tooth state numerical value and the first tooth state numerical value for each first tooth.

As an example, referring to FIG. 7, the numerical value of the difference of each of the six first teeth may be 2, 16, 1, 9, 11, or 7.

In step S424, the user terminal 20 may calculate a weight-reflected difference numerical value for each first tooth based on a difference numerical value for each first tooth and a weight for each first tooth.

According to the embodiment, the user terminal 20 may calculate a weight-reflected difference numerical value for each first tooth by adding weights for each first tooth to difference numerical value for each first tooth.

As an example, referring to FIG. 7, the weight-reflected difference numerical values of the six first teeth may be w_{A}×2, w_{B}×16, w_{C}×1, w_{D}×9, w_{E}×11, and w_{F}×7, respectively.

In step S425, the user terminal 20 may calculate the tooth deterioration information based on the weight-reflected difference numerical values for each first tooth.

According to the embodiment, the user terminal 20 may calculate the tooth deterioration information by summing the weight-reflected difference numerical values for each first tooth.

As an example, referring to FIG. 7, the tooth deterioration information may be (w_{A}×2)+(w_{B}×16)+(w_{C}×1)+(w_{D}×9)+(w_{E}×11)+(w_{F}×7).

In other words, according to another embodiment of the step S40, teeth treated at a hospital may not be used for calculating the tooth deterioration numerical value. In addition, when calculating the tooth deterioration numerical value, by setting the weight of the tooth in the caries normal stage to be less than the weight of the tooth belonging to the caries attention stage and the caries risk stage, the tooth deterioration numerical value may accurately reflect the actual deterioration degree of user's teeth.

Referring back to FIG. 4, in step S50, the user terminal 20 may provide various types information related to the tooth deterioration resin information and/or the tooth management notification message guiding the user's tooth management to the user. The related information or the tooth management notification message may be displayed on or output to the touch display 230.

According to the embodiment, in step S50, the user terminal 20 may display the tooth deterioration resin information on the touch display 230. Accordingly, the user may easily check whether his or her teeth have deteriorated.

Further, according to the embodiment, in step S20, the tooth self-management information may be input at least once or more during the non-measurement period. In this case, in step S50, the user terminal 20 may calculate the user's tooth self-management performance rate in the non-measurement period based on the tooth self-management information input at least once or more. In step S50, the user terminal 20 may generate the tooth management notification message by combining the user's tooth self-management performance rate and tooth deterioration information.

As an example, when the tooth self-management performance rate is greater than the first threshold rate corresponding to the high level, and the tooth deterioration numerical value is greater than the first threshold value corresponding to the high level, the user terminal 20 may generate the tooth management notification message including the correct tooth self-management method and/or the genetic predisposition information for an occurrence of caries.

That is, when both the tooth self-management performance rate and the tooth deterioration numerical value are high, it may be inferred that the tooth state is greatly deteriorated even though the user has relatively well managed his or her teeth during the non-measurement period. The cause of this situation may be that a user performs wrong tooth management or user's teeth are genetically weak. Accordingly, the user terminal 20 may notify the user of the correct tooth self-management method or notify the user of information that the user's teeth are genetically weak.

As another example, when the tooth self-management performance rate is lower than the second threshold rate corresponding to the low level, and the tooth deterioration numerical value is greater than the first threshold value corresponding to the high level, the user terminal 20 may generate the tooth management notification message including information to encourage the user's tooth self-management.

That is, when the tooth self-management performance rate is low and the tooth deterioration numerical value is high, it may be inferred that a user does not perform his or her tooth management during the non-measurement period even though the tooth state is greatly deteriorated. Accordingly, the user terminal 20 may notify the user of information, “I did not perform tooth self-management properly. Please take care of your teeth more diligently”.

As another example, when the tooth self-management performance rate is greater than the first threshold rate corresponding to the high level, and the tooth deterioration numerical value is less than the second threshold value corresponding to the low level, the user terminal 20 may generate the tooth management notification message including the information that the user's tooth self-management is performed diligently.

That is, when the tooth self-management performance rate is high and the tooth deterioration numerical value is low, it may be inferred that the user relatively well manages his or her teeth during the non-measurement period and thus the tooth deterioration numerical value is low. Accordingly, the user terminal 20 may notify a user of information that “a state of teeth has been maintained by performing tooth self-management well”.

Meanwhile, a user may belong to the same target management group as at least one other user. As an example, the target management group may be a daycare center, a kindergarten, a nursing hospital, etc.

According to the embodiment, in step S50, the user terminal 20 may generate first comparison information obtained by comparing the tooth deterioration numerical value of each user belonging to the target management group with the user's tooth deterioration numerical value and provide the generated first comparison information to a user. In this way, the user's competitive spirit for the tooth management may be encouraged.

Here, the first comparison information may correspond to percentile information of the tooth deterioration numerical value of a user with respect to the tooth deterioration numerical values of each user belonging to the target management group. As an example, the user terminal 20 may provide a user with information that the user's tooth deterioration numerical value is the top 30% of the tooth deterioration numerical values of each user belonging to the target management group. However, the present invention is not limited thereto, and various types of information such as ranking information may be provided to a user. Also, the tooth deterioration numerical values of each user belonging to the target management group may be received by the tooth management server 30.

According to another embodiment, in step S50, the user terminal 20 may calculate second comparison information obtained by comparing the tooth deterioration information of the target management group with the tooth deterioration information of at least one other management group, and provide the calculated second comparison information to a user. In this way,, the tooth deterioration numerical values may be compared for each management group, and the user's competitive spirit for tooth management may be further encouraged.

Here, each of the tooth deterioration information of the target management group and at least one other management group may be calculated based on the tooth deterioration information of users belonging to the corresponding management group. For example, the tooth deterioration information of users belonging to the corresponding management group may be an average value of the tooth deterioration numerical values of the users belonging to the corresponding management group. In addition, the tooth deterioration numerical values of other management groups may be received by the tooth management server 30.

Meanwhile, steps S20 to S50 may be repeatedly performed. That is, steps S20 to S50 may be performed periodically. In this case, the tooth state numerical value of the previous period may be the tooth state numerical value of the current period.

In addition, embodiments of the present invention may be implemented in a form of program commands that may be executed through various computer means and may be recorded in a computer-readable recording medium. The computer-readable recording medium may include program commands, data files, data structures or the like, alone or a combination thereof. The program commands recorded in the computer-readable recording medium may be especially designed and configured for the present invention or be known to those skilled in a field of computer software. Examples of the program commands include a high-level language code capable of being executed by a computer using an interpreter, or the like, as well as a machine language code made by a compiler. The above-described hardware device may be constituted to be operated as one or more software modules to perform operations according to embodiments of the present invention, and vice versa.

Hereinafter, embodiments of the present invention will be described in detail with reference to the accompanying drawings.

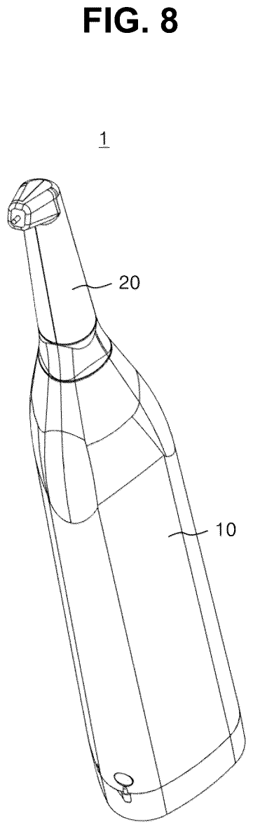

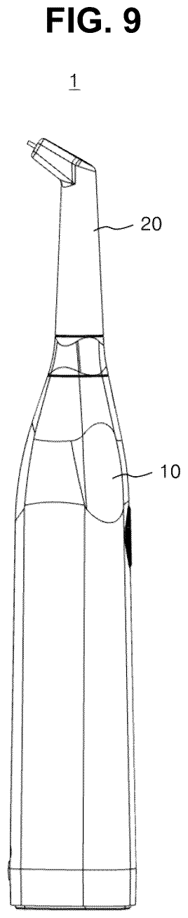

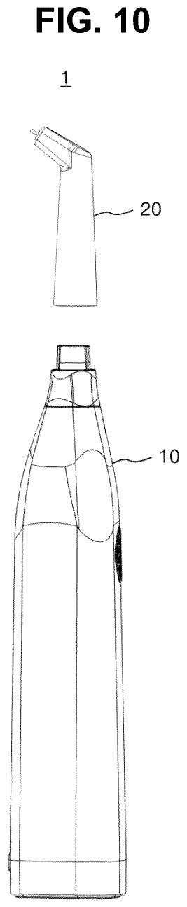

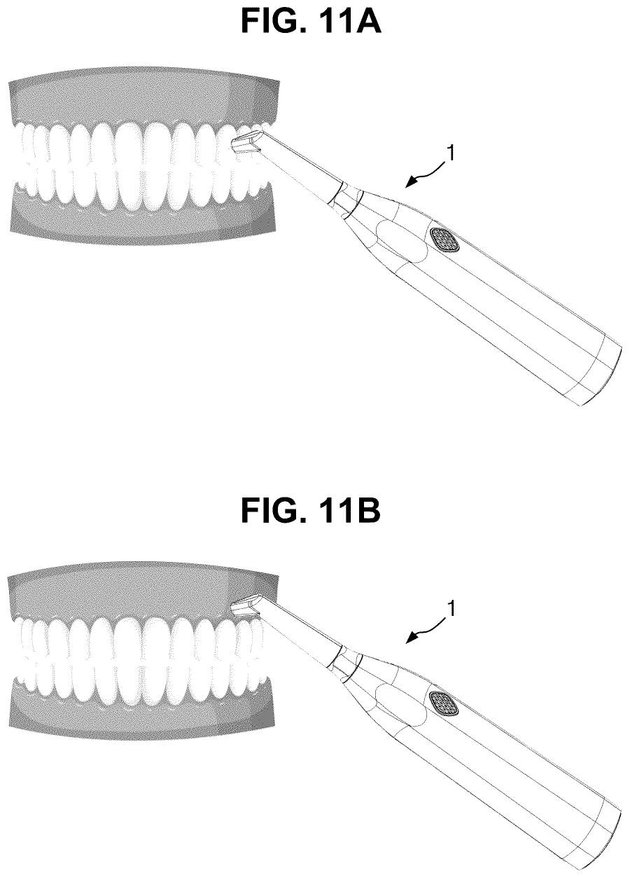

FIG. 8 is a perspective view of an oral diagnostic device 1 according to an embodiment of the present invention, FIG. 9 is a front view of the oral diagnosis device 1 according to the embodiment of the present invention, FIG. 10 is an exploded perspective view of the oral diagnosis device 1 according to the embodiment of the present invention, and FIGS. 11A and 11B are diagrams illustrating a state in which the oral diagnosis device 1 according to the embodiment of the present invention is used.

Referring to FIGS. 8 to 11B, the oral diagnostic device 1 according to an embodiment of the present invention may be a portable or mobile diagnostic device and may include a device main body 10 and a probe 20.

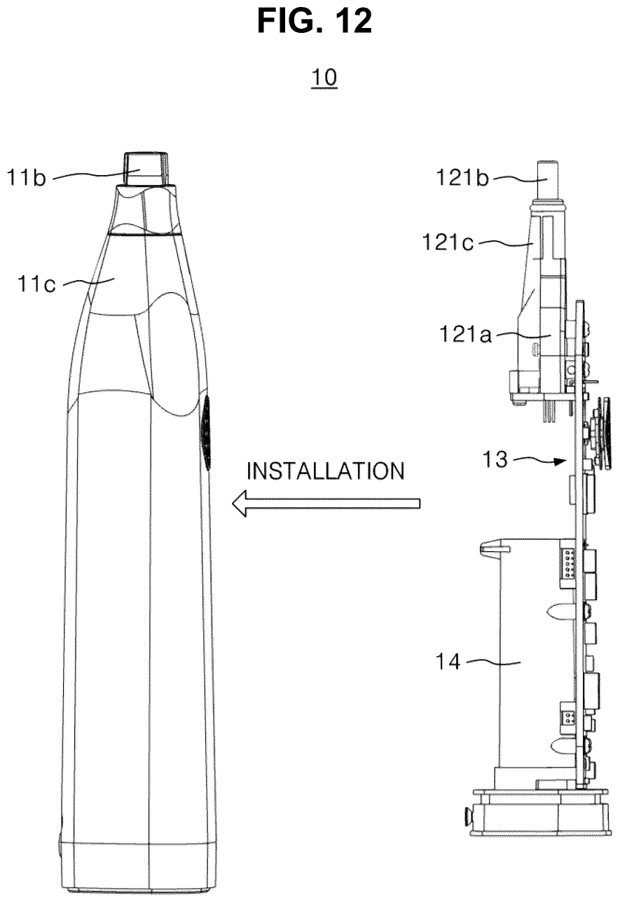

The device main body 10 is a main component of the oral diagnostic device 1, which generates light and may perform a function of diagnosing a condition of an oral cavity by analyzing light reflected from the oral cavity including teeth, gums, etc. As will be described later, in order to generate and receive light, the oral diagnostic device 1 may include an optical transmitting and receiving module 12 (FIGS. 12 to 15, and 17).

The probe 20 may be detachably coupled to the device main body 10, irradiate irradiated light generated and received from the optical transmitting and receiving module 12 to the oral cavity, and receive the reflected light from the irradiated light reflected from the oral cavity and transmit the received reflected light to the optical transmitting and receiving device 12. By being detachably coupled to the device main body 10, the probe 20 is replaceable.

Referring to FIGS. 11A and 11B, a user may hold the device main body 10 by hand and then allow the probe 20 to be disposed in contact with or very close to the teeth or gums. Accordingly, the oral diagnostic device 10 may diagnose or measure the condition of the oral cavity, such as the condition of the teeth or gums.

Hereinafter, the detailed configuration of the device main body 10 and the probe 20 will be described in more detail.

FIG. 12 is a diagram illustrating a detailed configuration of the device main body 10 according to the embodiment of the present invention.

Referring to FIG. 12, the device main body 10 may include a main body case 11, an optical transmitting and receiving module 12, a PCB module 13, and a battery module 14.

The main body case 11 may form the overall appearance of the device main body 10. The main body case 11 has an internal space, and the optical transmitting and receiving module 12, the PCB module 13, and the battery module 14 may be installed or accommodated in the internal space of the main body case 11.

The main body case 11 may include a first portion 11a, a second portion 11b, and a third portion 11c.

The first portion 11a of the main body case 11 may be held by the user's hand. A portion of the PCB module 13 and the battery module 14 may be accommodated in the internal space of the main body case 11 corresponding to the first portion 11a of the main body case 11.

A second portion 11b of the main body case 11 may be detachably coupled to the probe 20. A third portion 11c of the main body case 11 may connect the first portion 11a and the second portion 11b of the main case 11. The optical transmitting and receiving module 12 and the remaining portion of the PCB module 13 may be accommodated in the internal space of the main body case 11 corresponding to the second portion 11b and the third portion 11c of the main body case 11.

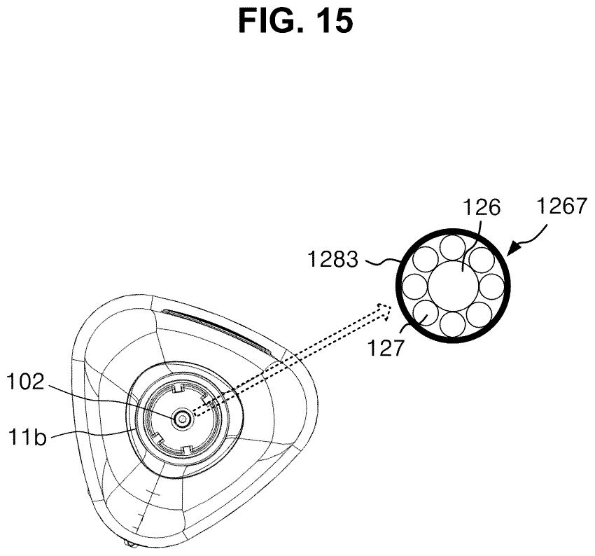

The second portion 11b of the main body case 11 may be formed to protrude from the first portion 11a of the main body case 11 through the third portion 11c of the main body case 11. Referring to FIG. 15, which will be described later, an opening 102 may be formed on an upper surface (one side surface) of the second portion 11b of the main body case 11.

The first portion 11c of the main body case 11 may generally have a shape of a triangular pillar. The third portion 11c of the main body case 11 may generally have a shape of a triangular pyramid. That is, corners constituting the outer surface of the third portion 11c of the main case 11 may be formed to be inclined at a specific angle.

The optical transmitting and receiving module 12 may generate the irradiated light and transmit the generated irradiated light to the probe 20, and detect the reflected light received from the probe 20 and transmit the detected reflected light to the PCB module 13. The detailed shape of the optical transmitting and receiving module 12 will be described later.

The PCB module 13 may control the optical transmitting and receiving module 12. In particular, the PCB module 13 may analyze the reflected light to diagnose or measure the oral cavity condition. The PCB module 13 may be configured to include a processor, a communication unit, etc. The PCB module 13 may transmit the diagnosed oral cavity condition to the user's terminal device, for example, a smart device through the communication unit.

The battery module 14 may supply driving power to the optical transmitting and receiving module 12 and the PCB module 13. The battery module 14 may be configured to include a dry cell and a battery compartment, or may be a battery that can be recharged using commercial power.

Hereinafter, the optical transmitting and receiving module 12 will be described in more detail.

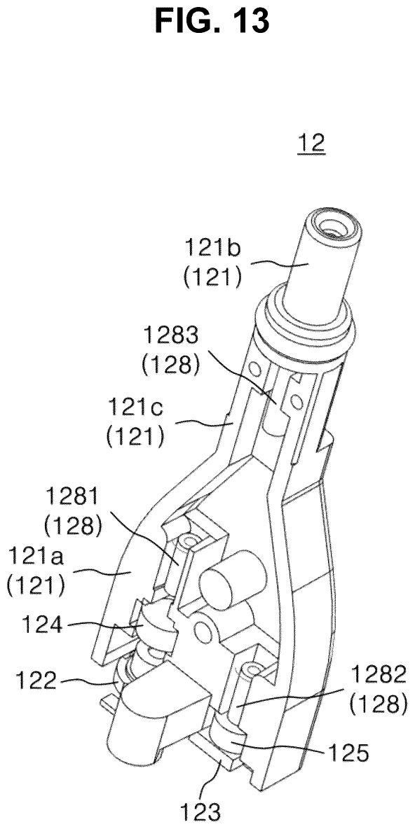

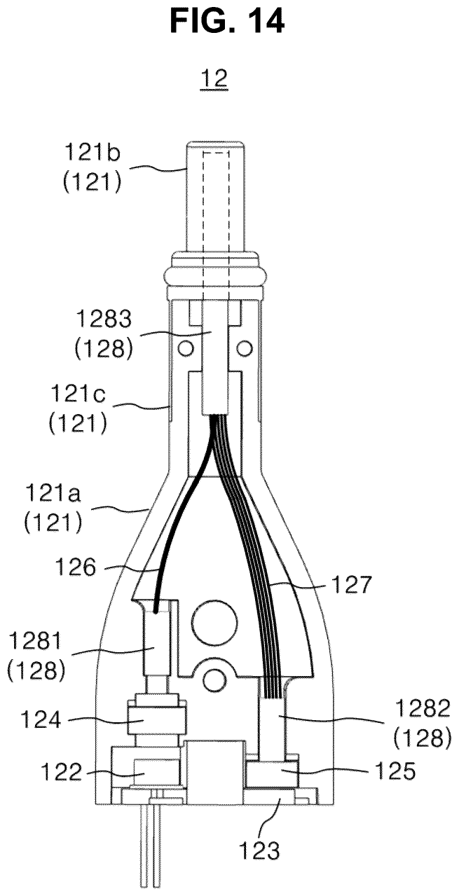

FIG. 13 is a perspective view of the optical transmitting and receiving module 12 according to an embodiment of the present invention, FIG. 14 is a cross-sectional view of the optical transmission/reception module 12 according to the embodiment of the present invention, and FIG. 15 is a diagram illustrating a plan shape of the optical transmitting and receiving module 12 installed in the main body case 11 according to an embodiment of the present invention. Meanwhile, for convenience of description, some components included in the optical transmitting and receiving module 12 are omitted in FIG. 13.

Referring to FIGS. 13 and 15, the optical transmitting and receiving module 12 may include a module case 121, a light-emitting element 122, a light-receiving element 123, a first lens 124, a second lens 125, a single first optical fiber 126, a plurality of second optical fibers 127, and an optical fiber tube 128. The light-emitting element 122 and the light-receiving element 123 may constitute optical element modules 122 and 123, and the optical fiber may also be called an optical cable.

The module case 121 may configure the exterior of the optical transmitting and receiving module 12. The module case 121 has an internal space, and optical element modules 122 and 123, a lens 124, an optical filter 125, a single first optical fiber 126, a plurality of second optical fibers 127, and an optical fiber tube 128 may be installed or accommodated in the internal space of the module case 121.

The module case 121 may include a first portion 121a, a second portion 121b, and a third portion 121c.

The optical element modules 122 and 123, one side of the single first optical fiber 126, and one side of the plurality of second optical fibers 127 may be disposed in the first portion 121a of the module case 121. The first portion 121a of the module case 121 may have both side surfaces of a convex curved shape. Referring to FIG. 12, the first portion 121a of the module case 121 may be accommodated in the third portion 11c of the main body case 11. In particular, since the third portion 11c of the main body case 11 has a shape of a triangular pyramid, the first portion 121a of the module case 121, which has both side surfaces of the convex curved shape, may be easily accommodated.

The second portion 121b of the module case 121 may be a portion of the module case 121 coupled to the probe 20, and the other side of the single first optical fiber 126 and the other side of the plurality of second optical fibers 127 may be disposed. Referring to FIG. 15, the other side of the single first optical fiber 126 and the other side of the plurality of second optical fibers 127 may be coupled to each other by an adhesive or the like to form an optical fiber coupling part 1267, and the optical fiber coupling part 1267 may be accommodated in the second portion 121b of the module case 121. The second portion 121b of the module case 121 may have a cylindrical shape.

Referring to FIGS. 12 and 15, the second portion 121b of the module case 121 may be accommodated in the second portion 11b of the main body case 11. In particular, an upper surface of the second portion 121b of the module case 121 may be disposed directly below the opening 102 formed in the second portion 11b of the main body case 11.

The third portion 121c of the module case 121 may connect the first portion 121a and the second portion 11b of the module case 121. A middle side of the single first optical fiber 126 and a middle side of the plurality of second optical fibers 127 may be disposed in the third portion 121c of the module case 121. The third portion 121c of the module case 121 may have a cylindrical shape. The second portion 121b of the module case 121 may be formed to protrude from the first portion 121a of the module case 121 through the third portion 121c of the main body case 121.

The light-emitting element 122 may generate the irradiated light. The light-emitting element 122 may be disposed along a first side surface of the first portion 121a of the module case 121. For example, the light-emitting element may be a laser diode that converts an electrical signal into light.

The light-receiving element 123 may detect the reflected light. The light-receiving element 123 may be disposed along a second side surface of the first portion 121a of the module case 121. For example, the light-receiving element 123 may be a photodiode that converts light into an electrical signal.

The lens 124 may focus the irradiated light generated from the light-emitting element 122. The lens 124 may be disposed along the first side surface of the first portion 121a of the module case 121.

The optical filter 125 may be a device that selectively transmits or prevents a band of a certain wavelength from being transmitted among the incident reflected light. The optical filter 125 may be disposed along the second side surface of the first portion 121a of the module case 121.

The single first optical fiber 126 may transmit the irradiated light generated by the light-emitting element 122 to the probe 20. One side of the single first optical fiber 126 may be disposed on the light-emitting element 122 side, and the other side of the single first optical fiber 126 may be disposed on the probe 20 side. One side of the single first optical fiber 126 may be disposed along the first side surface of the first portion 121a of the module case 121. Each of one side and the other side of the single first optical fiber 126 may be accommodated in a first optical fiber tube 1281 and a third optical fiber tube 1283, so the disconnection, etc., may be prevented.

The plurality of second optical fibers 127 may transmit the reflected light received by the probe 20 to the light-receiving element 123. For example, the number of second optical fibers 127 may be eight. One side of the plurality of second optical fibers 127 may be disposed on the light-emitting element 123 side, and the other side of the plurality of second optical fibers 127 may be disposed on the probe 20 side. One side of the plurality of second optical fibers 127 may be disposed along the second side surface of the first portion 121a of the module case 121. Each of one side and the other side of the plurality of second optical fibers 127 may be accommodated in a second optical fiber tube 1282 and a third optical fiber tube 1283, so the disconnection, etc., may be prevented.

According to an embodiment, the cross-section of the single first optical fiber 126 and the plurality of second optical fibers 127 may be circular, and the size (i.e., diameter) of the single first optical fiber 126 may be larger than that of each of the plurality of second optical fibers 127. For example, the size of the single first optical fiber 126 may be 0.5 π, and the size of each of the plurality of second optical fibers 127 may be 0.25 π.

According to an embodiment, one side of the single first optical fiber 126 and one side of the plurality of second optical fibers 127 may be disposed in a curved shape along both side surfaces of the first portion 121a of the module case 121. By disposing one side of the single first optical fiber 126 and the plurality of second optical fibers 127 in the curved shape, a transmission loss of the irradiated light and reflected light may be minimized. Meanwhile, in order to easily dispose one side of the single first optical fiber 126 and the plurality of second optical fibers 127 in the curved shape inside the optical transmitting and receiving module 12, the first portion 121a of the module case 121 may be formed to have both side surfaces in a convex curved shape.

As described above, referring to FIG. 15, the other side of the single first optical fiber 126 and the other side of the plurality of second optical fibers 127 are coupled to each other inside the third optical fiber tube 1283, so an optical fiber coupling part 1267 may be formed.

According to an embodiment, the optical fiber coupling part 1267 may be composed of a center area and an edge area. The single first optical fiber 126 may be disposed in the center area of the optical fiber coupling part 1267, and the plurality of second optical fibers 127 may be disposed in the edge area of the optical fiber coupling part 1267. In particular, the plurality of second optical fibers 127 may be disposed to surround the single first optical fiber 126.

Hereinafter, the probe 20 will be described in more detail.

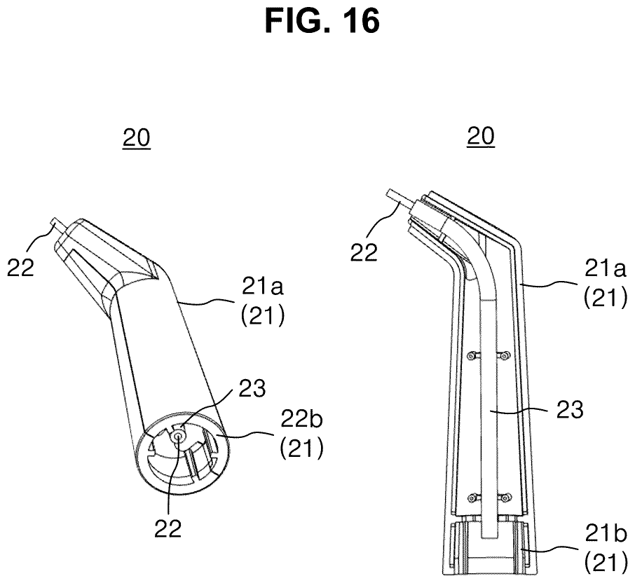

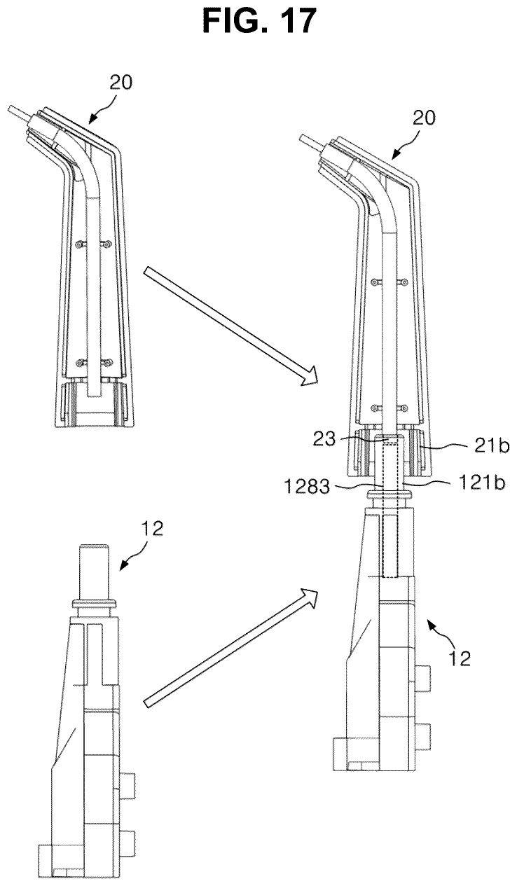

FIG. 16 is a perspective view of the probe 20 according to an embodiment of the present invention, and FIG. 17 is a diagram illustrating a combined shape of the optical transmitting and receiving module 12 and the probe 20 according to the embodiment of the present invention. Meanwhile, for convenience of description, the shape of the main body case 11 is omitted in FIG. 17.

Referring to FIG. 16, the probe 20 may include a probe case 21, a single third optical fiber 22, and a covering member 23.

The probe case 21 may configure the exterior of the probe 20. The probe case 21 has an internal space, and the single third optical fiber 22 may be accommodated in the internal space of the probe case 21.

The single third optical fiber 22 may irradiate the irradiated light received from the optical transmitting and receiving module 12 to the oral cavity, and receive reflected light from the irradiated light reflected from the oral cavity.

According to an embodiment, the cross-section of the single third optical fiber 22 may be circular, and the size (i.e., diameter) of the single third optical fiber 22 may be larger than that of the single first optical fiber 126 and that of each of the plurality of second optical fibers 127. As an example, the size of the single third optical fiber 22 may be 1.0 π.

Meanwhile, the probe case 21 may include a first portion 21a and a second portion 21b.

The middle side of the single third optical fiber 22 may be accommodated in the first portion 21a of the probe case 21.

One side of the single third optical fiber 22 may be accommodated in the second portion 21b of the probe case 21. Here, one side of the single third optical fiber 22 may be disposed to protrude from a portion of the internal space of the second portion 21b of the probe case 21. Referring to FIG. 17, one side of the single third optical fiber 22 may be disposed adjacent to the optical fiber coupling part 1256.

The other side of the single third optical fiber 22 may be disposed to protrude outward from the first portion 21a of the probe case 21. The other side of the single third optical fiber 22 may irradiate the irradiated light to the oral cavity and receive the reflected light reflected from the oral cavity.

The covering member 23 may be formed to surround one side and the middle side of the single third optical fiber 22 and protect one side and the middle side of the single third optical fiber 22.

Referring to FIG. 17, the second portion 21b of the probe case 21 may have the internal space corresponding to the shape of the second portion 11b of the main case 11. Accordingly, an inner surface of the second portion 21b of the probe case 21 is coupled with an outer surface of the second portion 11b of the main case 11 in the fitting shape, so the probe 20 may be coupled to the main case 11.

Referring to FIG. 17, when the probe 20 is not coupled to the second portion 11b of the main body case 11, one side of the single third optical fiber 22 may be disposed to protrude from the internal space of the second portion 21b of the probe case 21. Alternatively, when the probe 20 is coupled with the second portion 11b of the main body case 11, one side of the single third optical fiber 22 may pass through the opening 102 to be disposed adjacent to the optical fiber coupling part 1256 inside the second portion of the main body case 11.

In summary, the oral diagnostic device 1 according to an embodiment of the present invention may diagnose the condition of the oral cavity by transmitting and receiving light, for example, a laser, to the oral cavity. In this case, the optical fiber coupling part 1267 may be formed so that the single first optical fiber 126 transmitting the irradiated light is disposed in the center, and the plurality of second optical fibers 127 transmitting the reflected light are disposed at the edge. For an example, the plurality of second optical fibers 127 may be disposed in a circular shape with the single first optical fiber 126 as the center. In addition, the size of each of the plurality of second optical fibers 127 may be formed to be smaller than that of the single first optical fiber 126. Accordingly, the reflected light reflected from the diagnosis object (teeth, gums) may be received without exception, so the oral cavity condition of the diagnosis object may be accurately diagnosed without error.

According to the present invention, it is possible to periodically collect user's teeth-related state information measured by a tooth diagnostic device, and provide simply and easily a tooth state to a user based on the periodically collected tooth information.

In addition, according to the present invention, it is possible to provide tooth-related meaningful notification information, in particular, deterioration numerical information on a tooth to a user based on a measured tooth state.

In addition, according to the present invention, it is possible to provide genetic predisposition information on the occurrence of caries to a user.

In addition, according to the present invention, it is possible to perform tooth management for each management group including a plurality of users, and provide comparison information related to the management group to the user. In particular, the management group may be daycare center, kindergarten, nursing hospital, etc. Accordingly, it is possible to encourage competitive spirit of tooth management for a plurality of management users, and efficiently perform the tooth management.

In addition, according to the present invention, it is possible to easily and accurately diagnose the current condition of the oral cavity including teeth, interdentals, gums, etc.

In addition, it should be understood that the effects of the present invention are not limited to the above-described effects, and include all effects that can be inferred from the configuration of the invention described in the detailed description or claims of the present invention.

As described above, in the present invention, although specific matters such as detailed components and the like have been described with reference to limited embodiments and drawings, they have been provided only for assisting in the entire understanding of the present invention. Therefore, the present invention is not limited to the embodiments. Various modifications and changes may be made by those skilled in the art to which the present invention pertains from this description. Therefore, the spirit of the present invention should not be limited to these embodiments, but the claims described below and all of modifications equal or equivalent to the claims are intended to fall within the scope and spirit of the present invention.

What is claimed is:

1. A method for diagnosing conditions of teeth using an oral diagnostic device, the method comprising:
irradiating light, which is generated by an optical transmitting member of the oral diagnostic device, to each tooth of a user;
receiving, by an optical receiving member of the oral diagnostic device, the light reflected from the each tooth;
determining a first tooth state numerical value for the each tooth of the user based on first information associated with the each tooth, wherein the first information is obtained based on the light reflected from the each tooth during a first measurement period;
receiving tooth self-management information from the user at least once during a non-measurement period between the first measurement period and a second measurement period, which is after the first measurement period;
calculating tooth self-management performance rating for the user in the non-measurement period based on the received tooth self-management information;
determining a second tooth state numerical value for the each tooth of the user based on second information associated with the each tooth of the user, wherein the second information is obtained based on the light reflected from the each tooth during the second measurement period using the oral diagnostic device;
selecting at least one target tooth which was not treated in the non-measurement period;
calculating a difference between the second tooth state numerical value and the first tooth state numerical value for each of the at least one target tooth;
calculating a weighted difference for each of the at least one target tooth based on the difference and a weight assigned for each of the at least one target tooth; and
obtaining tooth deterioration information of the user at the second measurement period based on the weighted difference for each of the at least one target tooth.

2. The method of claim 1, wherein in said step of calculating a weighted difference, the weighted difference is calculated for each of the at least one target tooth by multiplying the weight assigned thereto with the difference between the second tooth state numerical value and the first tooth state numerical value, and
wherein the tooth deterioration information of the user corresponds to a sum of the weighted differences for all of the at least one target tooth.

3. The method of claim 1, wherein the weight for each of the at least one target tooth is set to, based on the second tooth state numerical value:
a first weight in response to the second tooth state numerical value being less than a first threshold, wherein the first threshold corresponds to a minimum tooth state numerical value at which enamel of teeth is damaged;
a second weight, which is greater than the first weight, in response to the second tooth state numerical value being equal to or greater than the first threshold and less than a second threshold, wherein the second threshold corresponds to a minimum tooth state numerical value at which dentin of teeth is damaged; and
a third weight, which is greater than the second weight, in response to the second tooth state numerical value being equal to or greater than the second threshold.

4. The method of claim 1, further comprising:
generating a tooth management notification message for guiding the user's tooth management by combining the tooth self-management performance rating with a tooth deterioration numerical value,
wherein the tooth deterioration numerical value is determined based on the tooth deterioration information.

5. The method of claim 4, wherein, in response to the tooth self-management performance rating being greater than a first threshold rating corresponding to a high level and the tooth deterioration numerical value being greater than a first threshold value corresponding to the high level, the tooth management notification message includes a correct tooth self-management method and/or genetic predisposition information for an occurrence of caries.

6. The method of claim 4, wherein, in response to the tooth self-management performance rating being less than a second threshold rating corresponding to a low level and the tooth deterioration numerical value being greater than a first threshold value corresponding to a high level, the tooth management notification message includes information to encourage the user's tooth self-management.

7. The method of claim 4, wherein, in response to the tooth self-management performance rating being greater than a first threshold rating corresponding to a high level and the tooth deterioration numerical value being lower than a second threshold value corresponding to a low level, the tooth management notification message includes information that the user has performed tooth self-management diligently.

8. The method of claim 1, further comprising:
generating first comparison information obtained by comparing tooth deterioration information of each user belonging to a target management group to which the user belongs with the tooth deterioration information of the user; and
providing the generated first comparison information.

9. The method of claim 8, wherein the first comparison information corresponds to percentile information of the tooth deterioration information of the user with respect to the tooth deterioration information of each user belonging to the target management group.

10. The method of claim 8, further comprising:
calculating second comparison information obtained by comparing tooth deterioration information of the target management group with tooth deterioration information of at least one other management group; and
providing the calculated second comparison information,
wherein each of the tooth deterioration information of the target management group and the at least one other management group is calculated based on tooth deterioration information of users belonging to the corresponding management group.

11. The method of claim 1, wherein said step of selecting at least one target tooth which was not treated in the non-measurement period includes receiving an input from the user.

12. The method of claim 1, wherein said step of selecting at least one target tooth which was not treated in the non-measurement period is performed based on comparison between the first tooth state numerical value and the second tooth state numerical value.

13. A terminal device, comprising:
a memory storing program instructions; and
a processor, when the program instructions are executed, configured to calculate tooth deterioration information according to the method of claim 1,
wherein the terminal device is operatively coupled with the oral diagnosis device.
